(19) United States
(12) Patent Application Publication
(10) Pub. No.: US 2007/0087060 A1
(43) Pub. Date: Apr. 19, 2007

(54) Ultrasound coupling medium for use in medical diagnostics

(76) Inventors: Rene H. Dietrich, Landschlacht (CH); Manfred Kreusch, Frankfurt (DE)

Correspondence Address: SPECKMAN LAW GROUP PLLC, 1201 THIRD AVENUE, SUITE 330, SEATTLE, WA, 98101, US

(21) Appl. No.: 10/557,913

(22) PCT Filed: May 21, 2004
(86) PCT No.: PCT/CH04/00308

§ 371 Date: Sep. 21, 2006

(30) Foreign Application Priority Data
May 21, 2003 (CH) 906/03

(51) Int. Cl. A61K 9/14

(52) U.S. Cl. 424/486; 424/488

(57) ABSTRACT

A composition of an ultrasound coupling medium is provided. The composition comprises at least 90% water, at least one preservative, and at least one base substance, wherein the composition is extensible into a film with a thickness of up to 1/10 mm, wherein the composition can withstand a pressure of up to 30 kp without tearing, wherein the composition can adapt exactly to skin surface without causing any significant air pockets, and wherein the composition can be removed from skin with substantially no residue left behind. The at least one base substance may be a galactomannan, a polyvinyl alcohol (PVA), a complex formation of galactomannan and borate ions, or comninations thereof.

### CROSS REFERENCE TO RELATED APPLICATIONS

This application claims priority under 35 U.S.C. §119(a) to Swiss Patent Application No. 906/03, filed May 21, 2003, under 35 U.S.C. §365(a) to International Patent Application No. PCT/CH2004/000308, filed May 21, 2004, and under 35 U.S.C. §120 to International Patent Application No. PCT/CH2004/000308, filed May 21, 2004 and Swiss Patent Application No. 906/03, filed May 21, 2003.

### FIELD OF THE INVENTION

The present invention relates to a composition of an ultrasound coupling medium for use in medical diagnostics, methods for applying the ultrasound coupling medium composition, and a package for the ultrasound coupling medium composition.

### BACKGROUND OF THE INVENTION

Conventional ultrasound coupling media known in the art are generally in the form of oily-viscous or pasty contact gels, based on starch or synthetic polymers, which can leave sticky and greasy residues on the head of ultrasound devices and on the patients' skin. These ultrasound coupling media are generally only suitable for use on patients having smooth skin or having little or no hair on the skin. The smearing of the ultrasound coupling medium on the patients' skin and the sound head of the ultrasound device can be unpleasant, and the sticky residue can cause formation of small air pockets on the patients' skin, which can compromise the quality of a sonogram. Also, removal of the ultrasound coupling medium from the patients' skin and the sound head entails additional cleaning efforts and expenses. Further, contamination of various parts of the ultrasound device, such as the device's keypad, can take place due to the unintentional transfer of the ultrasound coupling medium. In addition, these types of ultrasound coupling media may not be used on patients having bums, skin injuries, diseased skin changes, or sensitive skin. Some examples of these types of ultrasound coupling media include: mixtures of water, propylene glycol, acrylamide, acrylate, copolymer, and additives.

German Patent Application No. DE 35 26 874 discloses an ultrasonic conductive cushion constructed of a viscous, elastic and pressure-deformable material, which may be swelled with fluid before a sonography procedure. The thick cushion adapts to the contour of the surface to be examined, and it accommodates the pressure of the ultrasound measurement head, so that a patient's pressure-sensitive body locations may be examined. The fluid used for the swelling of the cushion is not permanently held by the viscous material. Thus, the fluid moistens the patient's skin in an area beneath the cushion and after the cushion is removed, the fluid is left behind on the patient's skin or it evaporates after a period of time.

German Patent Application No. DE-C-195 09 004 discloses a coupling cushion, for use with acoustic therapy devices, having the shape of an elastically yielding polymethyl pentene cover. The cushion is filled with a suitable propagation medium, such as water. The coupling cushion may also be manufactured as solid bodies of suitable acoustic propagation media, such as polyacrylamide gels or synthetic rubber.

European Patent Application No. EP-A-1′195′167 discloses the use of a solid gel body as a coupling cushion for use with acoustic therapy devices, based on reaction mixtures of polyols and polyiscocyanates components as a coupling medium for transmitting acoustic waves from a sound source onto a patient's body. The polyol component consists of one or more polyols with hydroxyl numbers below 112 and other polyols and additives known in the art. The isocyanate number of the reaction mixture is between about 15 to 59.81 and the product of the isocyanate functionality and the functionality of the polyol component is at least 6.15. Such coupling cushions are not suitable for use with sound heads of acoustic diagnosis devices because the coupling cushions are very expensive and the exchange of the coupling cushion after each examination is very time-consuming. Also, since the rubber-like surfaces of the coupling cushions prevent smooth movement of the sound head on a patient's skin, the use of a creamy or pasty lubricant would be necessary.

Thus, it is desirable to provide an ultrasound coupling medium to overcome the aforementioned disadvantages and problems.

### SUMMARY OF THE INVENTION

The present invention provides a composition of an ultrasound coupling medium suitable for use with conventional ultrasound devices. The inventive ultrasound coupling medium is preferably semi-liquid, can be extracted into a 1/10 mm thick film, and can withstand a pressure of up to 30 kp without tearing. The medium can adapt exactly to the surface of the skin without causing any significant air pockets.

The inventive ultrasound coupling medium comprises a gel mass or a structure gel. The gel mass or structure gel has a particularly slow-flowing cohesive consistency and is “visco-elastic”. The structure gel comprises at least 90% of bonded water, at least one preservative, and at least one base substance. The at least one base substance may be a polysaccharide, preferably a galactomanna, such as guar seed meal, carob seed meal, polyvinyl alcohol (PVA), combinations thereof, and the like. The complex formation of galactomannans with borate ions, which form hardly soluble complexes, may also be used to formulate the inventive ultrasound coupling medium.

A polyvinyl alcohol may alternatively be used as a base substance in formulating the inventive ultrasound coupling medium by way of hydrolysis (saponification) of polyvinyl acetate. The hydroxyl groups of the polyvinyl alcohol chain can associate with other substances. For example, polyvinyl alcohol may be mixed with boric acid salts (polyvinyl alcohol-boric acid-didiol complex) to form the inventive ultrasound coupling medium. Alternatively, the polyvinyl alcohol may be precipitated from borax solutions with relatively low concentration. The viscosity of the polyvinyl alcohol solution may be increased up to the gelification step, depending on the concentration of the solution.

In operation, the inventive ultrasound coupling medium may be pressed out from a packaging, such as tubes, bags, and the like. The packaging is provided with the inventive medium and a cutting means. In one embodiment, the cutting means is a flat and pliable plastic strip having a first end in the region of the dispensing opening of the packaging. The cutting means may be fastened or integrally formed on the packaging. The plastic strip in the end region comprises an opening, which may be fastened onto a neck or a screw closure. A free front end of the plastic strip is preferably sharp-edged and may serve as a cutting means.

After the desired quantity of the inventive gel medium is dispensed, the cutting means permits the gel strand to be cut off hygienically and directly at the discharge location. Thus, the unused portion of the gel remains completely in the packaging and is prevented from contamination. In a preferred embodiment, the cutting means is pre-formed in a u-shaped or v-shaped manner and the cutting of the gel strand may be achieved with one hand. The ultrasound coupling gel medium dosed in such a manner may either be deposited directly onto the patient's skin or onto the sound head of the ultrasound device, to which the medium sticks in an adhesive manner.

### BRIEF DESCRIPTION OF THE DRAWINGS

The present invention will be described in greater detail in the following detailed description, with reference to the accompanying drawings, wherein:

FIG. 1 illustrates the viscous and elastic measurements of various formulations of the inventive ultrasound coupling medium; and

FIG. 2 illustrates the mechanical oscillation measurements of various formulations of the inventive ultrasound coupling medium.

### DETAILED DESCRIPTION OF THE INVENTION

The present invention provides a composition of an ultrasound coupling medium suitable for use with conventional ultrasound devices. The inventive ultrasound coupling medium is capable of ensuring a good sound transmission (contrast-rich echo picture).

The inventive ultrasound coupling medium is preferably semi-liquid, can be extracted into a 1/10 mm thick film, and can withstand a pressure of up to 30 kp without tearing. The medium can adapt exactly to the surface of the skin without causing any significant air pockets. Even when the inventive ultrasound coupling medium is applied to skin with a lot of hair, substantially no significant air pockets occur between the medium, the sound head of the ultrasound device, and the skin. The inventive ultrasound coupling medium can be removed from the skin without leaving behind any residue and shortly after it is removed, the feeling of moisture is quickly eliminated. The medium, having slight aseptic properties, may be kept for at least 1 year, and may be applied to patients having allergies and inflammation of the skin. Also, since the inventive medium, with comparable or improved sound properties, neither moistens the sound head nor a patient's skin, it does not have to be wiped away like other ultrasound coupling medium known it the art; it may simply be pulled away from the patient's skin and the sound head without leaving behind any residue.

The inventive ultrasound coupling medium comprises a gel mass or a structure gel. The gel mass or structure gel has a particularly slow-flowing cohesive consistency and is “visco-elastic”, which means it is an intrinsically viscous (pseudo-plastic) substance. Thus, the inventive ultrasound coupling medium may be extracted into a thin and ductile film, and it may be cut and reduced into any sizes and different pieces of the medium may be molded into one collective piece of medium.

The structure gel comprises at least 90% of bonded water, at least one preservative, and at least one base substance. Despite containing a high portion of bonded water, syneresis does not take place within the structure gel. The addition of the at least one preservative provides a broad spectrum of anti-bacterial effects against gram-negative and gram-positive bacteria, as well as protection from molds. The at least one preservative may include, but is not limited to, pHB-methylester, pHB-propylester, salts of pHB-methylester, salts of pHB-propylester, mixtures thereof, and the like.

The at least one base substance may be a polysaccharide, preferably a galactomanna, such as guar seed meal, carob seed meal, polyvinyl alcohol (PVA), combinations thereof, and the like. Galactomannan is a group of vegetable fibrils that are found as reserve carbohydrates mainly in the seeds of many leguminous plants. Guar gum is the common term for the ground endosperm of the guar bean, such as Cyamopsis tetragonoloba L. or Cyamopsis psoraloides DC. The vegetable macromolecules contain polymannose main chains with galactose side chains. The application possibilities of galactomannans in the field of general technological processes are very versatile. As a trade product, these hydrocolloids are mainly applied as gelling and thickening agents. The complex formation of galactomannans with borate ions, which form hardly soluble complexes, may also be used to formulate the inventive ultrasound coupling medium.

Polyvinyl alcohol (PVA) is commonly used for the manufacture of pharmaceutical emulsions, ointments, and cosmetics, such as facial masks and skin protection ointments. Since polyvinyl alcohols are polymers of vinyl alcohol, they cannot exist in free form. Thus, in embodiments where a polyvinyl alcohol is used as a base substance to formulate the inventive ultrasound coupling medium, the formulation process involves the hydrolysis (saponification) of polyvinyl acetate. Also, the polyvinyl alcohols used to formulate the present invention generally meet the legal requirements with respect to the degree of purity.

The regularly arranged hydroxyl groups of the polyvinyl alcohol chain can generally form chemically stable complex compounds or associate with certain substances. For example, polyvinyl alcohol may be mixed with boric acid salts (polyvinyl alcohol-boric acid-didiol complex) to form the inventive ultrasound coupling medium. Alternatively, the polyvinyl alcohol may be precipitated from borax solutions with relatively low concentration. The viscosity of the polyvinyl alcohol solution may be increased up to the gelification step, depending on the concentration of the solution.

It is known that solutions of two chemically different polymers in the same solvent, such as water, generally are not compatible with one another. The mixing of two non compatable polymers usually result in immediate or eventual phase separation. This phenomenon also applies to mixtures of PVA-solutions with, for example, solutions of starch. Compatibility can be achieved most likely in the range of small portions of one component. Equal part mixtures are mostly poorly compatible with one another. Thus, for the composition of an embodiment of the inventive ultrasound coupling medium, it is essential that a good compatibility of two polymers is acheived, so that the characteristics described above for a suitable structure gel are fulfilled.

### EXAMPLES

PVA-guar gels:

In one embodiment, the inventive ultrasound coupling medium comprises at least one PVA with a hydrolysis degree (saponification degree) of at least 85 mole %. Preferably, fully-hydrolyzed PVA (hydrolysis degree of at least 98 mole %) is used. For example, the viscosity of the hydrolyzed PVA in a 4% aqueous solution (20° C.) is 30 mPa·s.

A good compatibility can be achieved by utilizing an alkylated galactomannan having a substitution degree DS of 0.2 to 0.6 (preferably hydroxypropyl-guar DS 0.3-0.5). The preferred viscosity of the hydroxypropyl guar in a 2% aqueous solution is 10,000-12,000 mPa·s. (20° C.).

Alternatively, the inventive ultrasound coupling medium comprises a hydoxyalkyl derivative. The hydoxyalkyl derivative may be manufactured by mixing a polysaccharide with an ethylene oxide or a propylene oxide in an alkaline medium.

Embodiment 1:

In this embodiment, the inventive ultrasound coupling medium comprises at least 90% water, and a mixture of about 1 to 5% by weight of galactomannan and about 0.3 to 5% by weight of PVA, having a pH-value of 6.5 to 8.5. For complex formation, sodium tetraborate (borax) and boric acid (0.1 -0.5% by weight) may be used.

The desired visco-elastic property is set on the basis of the mixing ratio of galactomanan and PVA, and by way of the addition of a suitable softening agent to achieve the interaction of the viscous and elastic components. Suitable softeners may be selected from the group consisting of: glycerol, ethyl glycol, diethylene glycol, triethylene glycol, PEG, and glycerin. In a preferred embodiment of the inventive ultrasound coupling medium, the desired “visco-elasticity” is set by way of the addition of 0.2 to 1.5% by weight of glycerol.

Some preferred compositions of the inventive ultrasound coupling medium utilizing PVA-guar gels are illustrated below:

Formulation a

| | |
|---|---|
| 1.60% by weight | hydoxypropyl guar (HPG) |
| 0.5% by weight | polyvinyl alcohol (PVA) |
| 97.0% by weight | water |
| 0.25% by weight | glycerol |
| 0.25% by weight | borax |
| 0.4% by weight | preservatives |
| <0.01% by weight | brilliant blue |

Formulation b

| | |
|---|---|
| 1.1% by weight | HPG |
| 3.9% by weight | PVA |
| 93.5% by weight | water |
| 0.8% by weight | glycerol |
| 0.35% by weight | borax |
| 0.35% by weight | preservatives |

Formulation c

| | |
|---|---|
| 3.0% by weight | HPG |
| 1.0% by weight | PVA |
| 93.5% by weight | water |
| 1.9% by weight | glycerol |
| 0.15% by weight | borax |
| 0.15% by weight | boric acid |
| 0.30% by weight | preservatives |

It was found that the advantages by applying the inventive ultrasound coupling medium, such as good film formation, achieving desirable elasticity and viscosity, non-stickiness to skin and sound head, and good sound transmission, can be achieved by combining cross-linking of guar gum, preferably hydroxypropyl guar and polyvinyl alcohol with borate ions.

Guar gels:

In another embodiment of the inventive ultrasound coupling medium, the coupling medium may be made based on a galactomannan/boron complex, without the addition of polyvinyl alcohol.

Embodiment 2:

Structure gels based on a galactomannan/boron complex with a galactomannan concentration of about 1 to 5% by weight, preferably about 3 to 4% by weight.

Formulation d

| | | |
|---|---|---|
| 3.7% | by weight | HPG |
| 94.0% | by weight | water |
| 1.7% | by weight | glycerol |
| 0.05% | by weight | borax |
| 0.25% | by eight | boric acid |
| 0.3% | by weight | preservatives |

Formulation e

| | |
|---|---|
| 2.0% by weight | HPG |
| 97.0% by weight | water |
| 0.4% by weight | glycerol |
| 0.05% by weight | borax |
| 0.2% by weight | boric acid |
| 0.35% by weight | preservatives |

Formulation f

| | |
|---|---|
| 1.0% by weight | guar gum |
| 98.0% by weight | water |
| 0.25% by weight | glycerol |
| 0.20% by weight | borax |
| 0.20% by weight | boric acid |
| 0.35% by weight | preservatives |

PVA gels:

In yet another embodiment, the inventive ultrasound coupling medium comprises a polyvinyl alcohol (PVA).

Embodiment 3:

Structure gels based on PVA having a PVA-concentration of about 4 to 8% by weight, preferably about 5 to 7% by weight.

Formulation g

| | |
|---|---|
| 5.0% by weight | PVA |
| 92.7% by weight | deionized water |
| 1.4% by weight | glycerol |
| 0.45% by weight | borax |
| 0.45% by weight | preservatives |

Formulation h

| | |
|---|---|
| 7.0% by weight | PVA |
| 90.0% by weight | deionized water |
| 2.0% by weight | glycerol |
| 0.4% by weight | borax |
| 0.6% by weight | preservatives |

Formulation i

| | |
|---|---|
| 6.0% by weight | PVA |
| 92.0% by weight | deionized water |
| 1.0% by weight | glycerol |
| 0.4% by weight | borax |
| 0.6% by weight | preservatives |

Preferably, the following products are used for the above mentioned formulations: Polyvinyl alcohol (PVA)—fully saponified CAS-No.: 9002-89-5, viscosity (4% solution/20° C.): 30 mPa·s (method: DIN 53015), pH-value (4% solution): 5.7 (method: ISO 1148); hydroxypropyl guar—DS 0.45; viscosity (2% solution): 11000 mPa·s (Brookfield RV).

In still another embodiment, colors, such as food coloring, may be added to the inventive ultrasound coupling medium. The colorings should meet purity specifications and the corresponding regulations. For example, brilliant blue FCF (E133, C.I. No. 42090) or Patent blue V (E131 C.I. No. 42051) is preferred for the inventive medium.

Material variables such as viscosity, elasticity, and creep test/creep recovery were measured for the inventive ultrasound coupling medium. A rheometer/vicsometer model CVO of the company Bohlin Instruments was used for the measurements. Measurement data for the gel formulations (a) and (b) from the group of the guar/PVA gels, gel formulation (d) from the group guar gum, and the gel formulation (g) from the group PVA are shown in the following table:

TABLE 1
Measurement values - creep test and creep recovery test

| Data: Formulation (d) | | Data: Formulation (a) | |
|---|---|---|---|
| summary creep test: | | summary creep test: | |
| start index: | 66 | start index: | 66 |
| end-index: | 75 | end-index: | 75 |
| viscosity: | 4.05e+03 Pas | viscosity: | 440 Pas |
| shear rate: | 0.00123 1/s | shear rate: | 0.0114 1/s |
| stationary: | 0.564 | stationary: | 0.721 |
| joc: | 0.0121 1/Pa | joc: | 0.0554 1/Pa |
| summary recovery test | | summary recovery test | |
| start index | 142 | start index | 142 |
| end index | 151 | end index | 151 |
| jor: | 0.0144 1/Pa | jor: | 0.0892 1/Pa |
| Data: Formulation (g) | | Data: Formulation (b) | |
| summary creep test: | | summary creep test: | |
| start index: | 66 | start index: | 66 |
| end-index: | 75 | end-index: | 75 |
| viscosity: | 246 Pas | viscosity: | 1.22e+03Pas |
| shear rate: | 0.0203 1/s | shear rate | 0.00409 1/s |
| stationary: | 0.973 | stationary: | 0.807 |
| joc: | 0.00793 1/Pa | joc: | 0.0124 1/Pa |
| summary recovery test: | | summary recovery test: | |
| start index | 142 | start index | 142 |
| end index | 151 | end index | 151 |
| jor: | 0.013 1/Pa | jor: | 0.014 1/Pa |

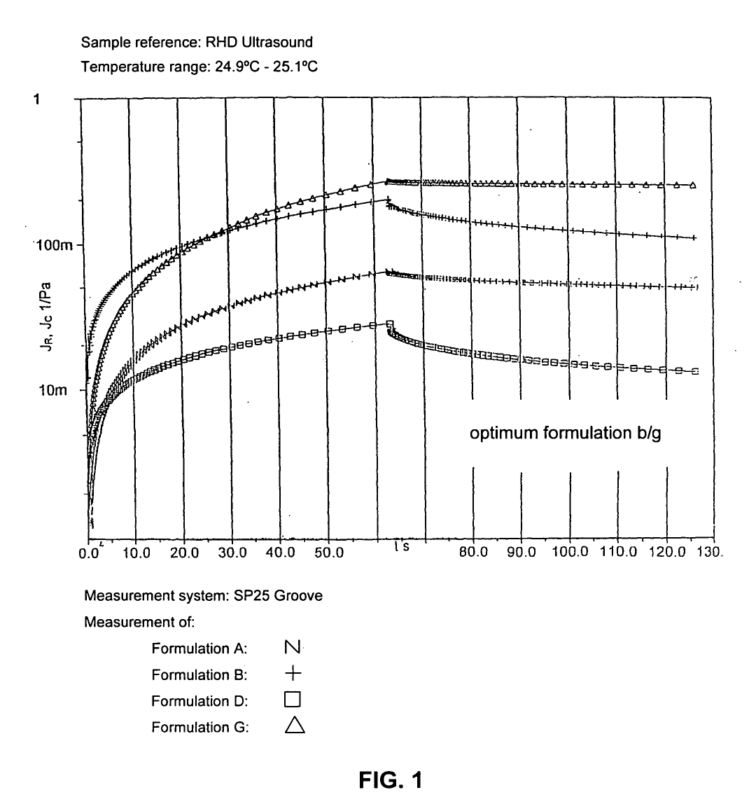

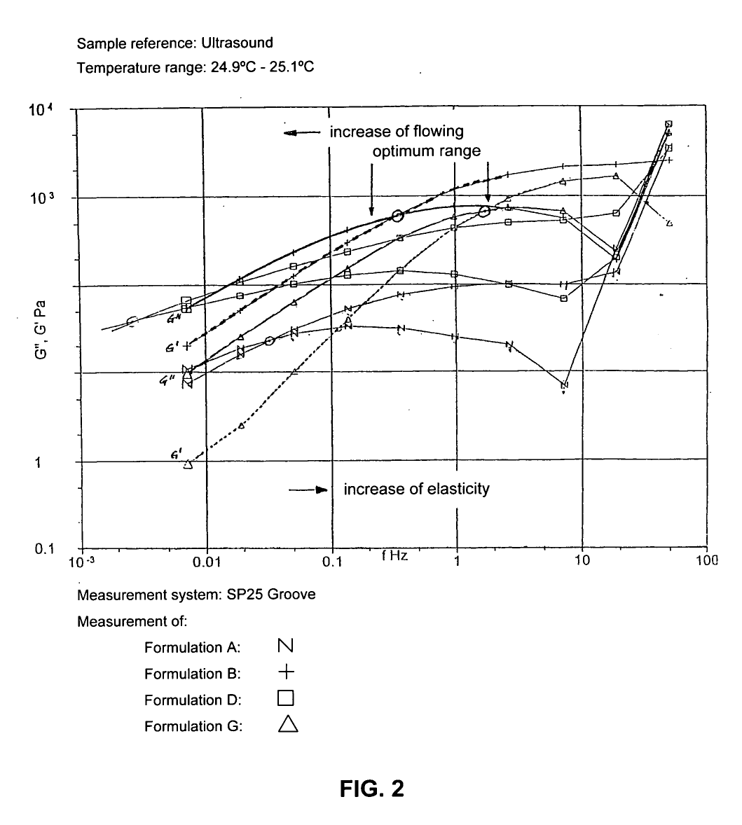

Measurement results of gel formulations (a) and (b) from the group of guar/PVA gels, gel formulation (d) from the group guar gum, and gel formulation (g) from the group PVA are additionally illustrated in FIGS. 1 and 2 by way of creep trials and mechanical oscillation measurements.

FIG. 1 illustrates the viscous and elastic measurements of gel formulations (a), (b), (d), and (g). The viscous and elastic components of the four gel formulations determined in a creep trial are shown. During this measurement, a constant shear stress is applied as a step function onto the formulations to be examined, their deformation is determined. The deformation divided by the applied shear stress results in the compliance J. In the graph illustrated in FIG. 1, J is plotted against time t in seconds. The unit of J as is shown in FIG. 1, is Pa⁻¹. The virtual shape of the curve obtained in the creep test is determined by the interaction of viscous and elastic components and serves for the characterization of the visco-elastic properties of the examined gel formulations. Compositions with a curved shape, such as gel formulations (b) and (g), are particularly preferred. The ratio of viscosity and elasticity is important for the optimal nature of the inventive ultrasound coupling medium.

FIG. 2 illustrates the mechanical oscillation measurements of gel formulations (a), (b), (d), and (g). In this measurement, a shear stress is applied to the four gel formulations to be examined in an oscillating manner. The deformation of the gel formulations is considered primarily as an answer signal. The memory modulus G′ describes the elastic behavior of the gel formulations and represents the recovered energy. The viscous behavior, respectively the dissipated energy of the gels, is computed and described in the known manner by the loss modulus G″. G′ and G″ are specified in Pa and are plotted against the applied angular frequency in Hz.

The intersection point of the memory modulus G′ with the loss modulus G″ is assumed as a measure of the gel point. Ideally the gel point for a composition according to the invention lies in an optimal region specified in FIG. 2, of 20 mHz to 5 Hz, wherein the region between 100 mHz and 1 Hz is particularly preferred.

Conventional measurements of viscosity in the region of more than 250,000 mPa·s are difficult to achieve and tend to have large measurement errors. Conventional measurements using a Brookfield RV (spindle 7, r.p.m 10, factor 4000; scale value 66.5; temperature 23° C.) with preferred formulations, viscosities in the desired optimal range around 266,000 mPa·s were determined.

Packaging with a Cutting Aid

In operation, the inventive ultrasound coupling medium may be pressed out from a packaging, such as tubes, bags, and the like, based on its pseudoelastic flow behavior. The packaging is provided with the inventive medium and a cutting means.

In one embodiment, the cutting means is a flat and pliable plastic strip having a first end in the region of the dispensing opening of the packaging. The cutting means may be fastened or integrally formed on the packaging. The plastic strip in the end region comprises an opening, which may be fastened onto a neck or a screw closure. A free front end of the plastic strip is preferably sharp-edged and may serve as a cutting means. The plastic strip preferably has a rectangular configuration and is wider that the dispensing opening. It is also preferred to be long enough in order for the cutting edge move completely past the dispensing opening.

After the desired quantity of the inventive gel medium is dispensed, the cutting means permits the gel strand to be cut off hygienically and directly at the discharge location. Thus, the unused portion of the gel remains completely in the packaging and is prevented from contamination. Also, since the cutting means is arranged directly at the dispensing opening, it is immediately available and will not be lost.

In a preferred embodiment, the cutting means is pre-formed in a u-shaped or v-shaped manner. Due to the shapes, the cutter points in the direction of the dispensing opening, and after the removal of the tube cap, the dispensing of the coupling gel medium, or the dosing and the cutting of the gel strand may be achieved with one hand. The ultrasound coupling gel medium dosed in such a manner may either be deposited directly onto the patient's skin or onto the sound head of the ultrasound device, to which the medium sticks in an adhesive manner. The medium may be extracted or extruded onto an actual film having a thickness of less than 1 mm. The medium may also be sized to fit various sizes of sound heads, ranging from about 0.5×1 cm (small sound heads) to about 1×7 to 8 cm (large sound heads). Depending on the sound head size and thickness of the patient's subcutaneous fat tissue, a greater pressure may be exerted onto the inventive gel medium during the examinations. The coupling gel medium extracted into a film is able to withstand a pressure of up to 30 kg without tearing.

In contrast to coupling cushions known in the art, the inventive ultrasound coupling medium is not surrounded by a casing or a membrane. If the known coupling cushions were pressed together to the above-mentioned thickness, then practically no ultrasound coupling medium can exist between the cushion membranes, and the ultrasound signals cannot be transmitted.

While certain embodiments of the present invention have been described, it will be understood that various changes may be made in the above invention without departing from the scope of the invention. It is intended that all matter contained in the above description or shown in the accompanying drawings shall be interpreted as illustrative and not in a limiting sense.

1-19. (canceled)

20. A composition of an ultrasound coupling medium, comprising:
(a) at least 90% water;
(b) at least one preservative; and
(c) at least one base substance,
wherein the composition is extensible into a film with a thickness of up to 1/10 mm, wherein the composition can withstand a pressure of up to 30 kp without tearing, wherein the composition can adapt exactly to skin surface without causing any significant air pockets, and wherein the composition can be removed from skin with substantially no residue left behind.

21. The composition of claim 20, wherein the at least one base substance is selected from the group consisting of: galactomannan, polyvinyl alcohol (PVA), a complex formation of galactomannan and borate ions, and combinations thereof.

22. The composition of claim 20, wherein the at least one base substance is a galactomannan and boron complex having a galactomannan concentration of 1-5% by weight, and is free of polyvinyl alcohol.

23. The composition of claim 20, wherein the at least one base substance is a galactomannan and boron complex having a galactomannan concentration of 3-4% by weight, and is free of polyvinyl alcohol.

24. The composition of claim 20, wherein the composition comprises 1 to 5 percent by weight of galactomannan, 0.3 to 5 percent by weight of polyvinyl alcohol, and 0.1 to 0.5 percent by weight of sodium tetraborate.

25. The composition of claim 24, wherein the composition has a pH value between 6.5 and 8.5.

26. The composition of claim 20, wherein the composition further comprises a softener selected from the group consisting of: ethyl glycol, di-ethylene glycol, tri-ethylene glycol, PEG, and glycerol.

27. The composition of claim 26, wherein the softener is 0.2 to 1.5% by weight of glycerol.

28. The composition of claim 20, wherein the gel point of the composition lies in a range from 20 mHz to 5 Hz.

29. The composition of claim 20, wherein the gel point of the composition lies in a range from 100 mHz and 1 Hz.

30. The composition of claim 20, wherein the at least one preservative is selected from the group consisting of pHB-methylester, pHB-propylester, salt of pHB-methyleste, salt of pHB-propylester, and mixtures thereof.

31. The composition of claim 20, wherein the composition further comprises colorings, selected from the group consisting of: Brilliant blue FCF (E133, C.I. No. 42090) and Patent blue V (E131 C.I. No. 42051).

32. The composition of claim 21, wherein the polyvinyl alcohol has a hydrolysis degree of at least 85 mole %.

33. The composition of claim 32, wherein the composition comprises a 4% aqueous PVA-solution with a viscosity of 30 mPa·s. (20° C.) and a 2% aqueous hydroxypropyl guar solution with a viscosity of 10,000 to 12,000 mPa·s. (20° C.).

34. The composition of claim 21, wherein the polyvinyl alcohol has a hydrolysis degree of at least 98 mole %.

35. The composition of claim 21, wherein the galactomannan is an alkylated galactomannan with a substitution degree DS of 0.2 to 0.6.

36. The composition of claim 21, wherein the galactomannan is an alkylated galactomannan with a substitution degree DS of 0.3 to 0.5.

37. A method for medical dialogistic by applying an ultrasound coupling medium, comprising:
(a) at least 90% water;
(b) at least one preservative; and
(c) at least one base substance selected from the group consisting of: galactomannan, polyvinyl alcohol (PVA), a complex formation of galactomannan and borate ions, and combinations thereof;
wherein the composition is extensible into a film with a thickness of up to 1/10 mm, wherein the composition can withstand a pressure of up to 30 kp without tearing, wherein the composition can adapt exactly to skin surface without causing any significant air pockets, and wherein the composition can be removed from skin with substantially no residue left behind.

38. A packaging of an ultrasound coupling medium comprising a composition and a cutting means for cutting off a gel strand, wherein the composition comprises at least 90% water, at least one preservative, at least one base substance selected from the group consisting of: galactomannan, polyvinyl alcohol (PVA), a complex formation of galactomannan and borate ions, and combinations thereof, wherein the packaging is provided with a reclosable dispensing opening, wherein the cutting means is arranged in the region of the dispensing opening, and wherein the cutting means is a flat and pliable plastic strip provided with a front edge formed into a cutter.

39. The packaging of claim 38, wherein the plastic strip has a rectangular configuration, wherein the plastic strip is wider than the dispensing opening, and wherein the plastic strip is long enough in order to lead the cutter edge past the dispensing opening.
